(19) United States
(12) Patent Application Publication
(10) Pub. No.: US 2025/0060350 A1
(43) Pub. Date: Feb. 20, 2025

(54) CONTAMINATED SITE SAMPLING ROBOT, AND INTELLIGENT SAMPLING METHOD

(71) Applicant: TIANJIN UNIVERSITY, Tianjin (CN)

(72) Inventors: Li HE, Tianjin (CN); Tong SUN, Tianjin (CN); Kaiwen CHENG, Tianjin (CN); Fubin ZHANG, Tianjin (CN); Haochuan Guo, Tianjin (CN)

(73) Assignee: TIANJIN UNIVERSITY, Tianjin (CN)

(21) Appl. No.: 18/805,533

(22) Filed: Aug. 15, 2024

(30) Foreign Application Priority Data
Aug. 17, 2023 (CN) 202311038100.9

(51) Int. Cl. G01N 33/24; G01N 1/08; G05D 1/689; G05D 105/80

(52) U.S. Cl. CPC G01N 33/24; G01N 1/08; G05D 1/689; G05D 2105/87; G05D 2105/89

(57) ABSTRACT

Disclosed is a contaminated site sampling robot and an intelligent sampling method thereof. The robot includes a body with a walking mechanism, vision sensing system, drilling mechanism, and negative-pressure suction mechanism. The walking mechanism features two servo motors within a mounting platform, with track wheels attached to the servo motors' output ends. The vision sensing system comprises a supporting frame at one end of the mounting platform, equipped with a vision sensing camera and radar sensor. The drilling mechanism consists of a U-shaped base on the mounting platform's top, with a mechanical arm and drilling machine mounted at one end. The negative-pressure suction mechanism includes a U-shaped box on the mounting platform's middle part, housing a vacuum cleaner whose dust outlet connects to a sample collecting box. This setup allows for optimal path identification, positioning, obstacle avoidance, and control of sampling conditions, facilitating intelligent sampling of contaminated sites.

### TECHNICAL FIELD

The present invention relates to the technical field of environmental pollution prevention, and in particular to a contaminated site sampling robot capable of implementing intelligent sampling and cooperative sampling, and an intelligent sampling method.

### BACKGROUND

The existing sampling operation for the soil of a contaminated site is the most time-consuming, laborious and costly part of the whole production process of the contaminated site. With the continuous development of robots and intelligent sampling, the application of a robot technology to the contaminated site sampling has become the general trend. For example, the 528LS direct-push type soil sampling machine is actually an intelligent soil sampling robot, with intelligent remote operation, accurate and agile hole adjustment, microwave high-frequency impact, efficient undisturbed sampling and wireless control apparatus controlled by an oil pressure apparatus. An operator can remotely operate a series of actions, such as operating a crawler of the sampling machine to walk, moving the machine body left and right, lifting the walking frame and performing accurate sampling. However, during use, the direct-push type soil sampling machine cannot arrive at a sampling site autonomously completely and cannot perform negative-pressure collection on samples.

### SUMMARY

An objective of the present invention is to solve the technical problems an existing sampling robot for a contaminated site cannot arrive at a sampling site autonomously completely and cannot perform negative-pressure collection on samples, and to provide a contaminated site sampling robot and an intelligent sampling method.

To solve the above-mentioned technical problems, the technical solution adopted by the present invention is as follows:
a contaminated site sampling robot includes a robot body, where the robot body includes a walking mechanism, a vision sensing system, a drilling mechanism and a negative-pressure suction mechanism;
the walking mechanism includes a mounting platform, two servo motors are fixedly mounted at two ends inside the mounting platform, a track wheel is mounted at an output end of each of the servo motors, and a walking track is sleeved on two track wheels located on a same side;
the vision sensing system includes a supporting frame mounted at the top of one end of the mounting platform, and a vision sensing camera and a radar sensor are arranged on one side of the top of the supporting frame;
the drilling mechanism includes a U-shaped base mounted at the top of the mounting platform, an interior of one end of the U-shaped base is hinged with a first supporting arm, the top of the first supporting arm is hinged with a second supporting arm, a telescopic arm is sleeved inside the second supporting arm, a rotating base is rotatably mounted at one end of the telescopic arm, and a drilling machine is mounted on one side of the rotating base; and
the negative-pressure suction mechanism includes a U-shaped box mounted at the top end of a middle part of the mounting platform, a vacuum cleaner is mounted inside the U-shaped box, a dust outlet of the vacuum cleaner is connected to a sample collecting box, and a feeding port of the vacuum cleaner is connected to a sample collecting pipe through a pipeline.

A supporting mechanism is fixedly mounted at the bottom of the supporting frame, the supporting mechanism includes two first electric telescopic rods fixedly mounted at the bottom of the supporting frame, and supporting pads are fixedly mounted at the bottoms of the first electric telescopic rods.

A battery box is fixedly mounted at the top of the other end of the mounting platform, a control box and a sample tank placing rack which are respectively located on two sides of the U-shaped box are further mounted at the top of the mounting platform, and a plurality of placing grooves are formed in a surface of the sample tank placing rack.

The vision sensing system further includes a gear motor and a lead screw base which are mounted at the tops of bosses at two ends of the supporting frame, a double-thread lead screw is mounted at an output end of the gear motor, an exterior of the double-thread lead screw is in threaded connection with two lead screw nuts, guide rods are connected to middle parts of the two lead screw nuts in an inserting manner, two ends of the guide rods are fixedly connected to two bosses in a middle part of the supporting frame, and the vision sensing camera and the radar sensor are fixedly mounted on one side of each of the two lead screw nuts, respectively.

The drilling mechanism further includes a second electric telescopic rod hinged at the bottom of the second supporting arm, the bottom of the second electric telescopic rod is hinged with the top of a boss at one end of the U-shaped base, a third electric telescopic rod is fixedly mounted at the top of the second supporting arm, one end of the third electric telescopic rod is hinged with the top of the rotating base, the bottom of one end of the rotating base is hinged with a fourth electric telescopic rod, and the fourth electric telescopic rod is mounted at the bottom of the second supporting arm away from a telescopic end.

The sample collecting box is also mounted inside the U-shaped box, a sample tank communicating with the dust outlet of the vacuum cleaner is arranged inside the sample collecting box, and the top of the sample collecting box is hinged with a box cover.

The pipeline is a corrugated pipe, and a middle part of the pipeline is placed inside the hollow telescopic arm.

A PLC is mounted inside the control box, the vision sensing camera and the radar sensor are electrically connected to the PLC, the PLC is electrically connected to the gear motor, the vacuum cleaner, the drilling machine, the first electric telescopic rod, the second electric telescopic rod, the third electric telescopic rod and the fourth electric telescopic rod, and the PLC is electrically connected to a battery inside the battery box and a direct-current generator.

An intelligent sampling method for a contaminated site uses the contaminated site sampling robot to collect soil, and specifically includes the following steps:
Step 1: performing basic information input, including: device parameter information input, site resource data docking and sampling target input, including the following substeps:
Step 1-1: endowing variables of the PLC, the vision sensing camera and the radar sensor as default values, and setting the gear motor, the vacuum cleaner, the drilling machine, the first electric telescopic rod, the second electric telescopic rod, the third electric telescopic rod and the fourth electric telescopic rod as default states;
Step 1-2: inputting site environment information and space coordinate distribution information; and
Step 1-3: determining a distribution mode of sampling areas of the contaminated site and a sampling quantity requirement of each of the sampling areas;
Step 2: performing preliminary work preparation, including sampling position collection, path optimization and device preheating, including the following substeps:
Step 2-1: surveying, by the robot, a site environment, and acquiring a terrain, an obstacle position and air quality information of the site by a road condition vision sensor to provide reference for the subsequent navigation and sampling;
Step 2-2: performing, by the robot, self-positioning and surrounding environment perception by a radar sensor, constructing a map by an SLAM technology and updating the own position, considering, by a control unit, whether factors such as a maximum corner and a maximum power stroke of the robot are met according to a preset robot starting point and a target sampling point and in combination with map data and obstacle information, and determining a best path to a sampling point by an intelligent sampling algorithm, where a robot path planning fitness function is:

F=Σᵢ₌₁ⁿ(ω₁·lᵢ+ω₂·hᵢ+ω₃·αᵢ)+(ωₐ·fₐ+ωₛ·fₛ+ωₗ·fₗ)

in the formula, lᵢ is the length of a i^{th} reference path, hᵢ is the height difference of the i^{th} reference path, αᵢ is the smoothness of the i^{th} reference path, ω₁, ω₂ and ω₃ are respectively weights of the length, the height difference and the smoothness, f_{α} indicates whether the steering angle of the reference path exceeds a maximum steering angle, the value is 1 if the steering angle of the reference path exceeds the maximum steering angle, and the value is 0 if the steering angle of the reference path does not exceed the maximum steering angle, fₛ indicates whether the reference path exceeds a power range, the value is 1 if the reference path exceeds the power range, and the value is 0 if the reference path does not exceed the power range, fi indicates whether a reference track intersects with an obstacle in a task area, the value is 1 if the reference track intersects with the obstacle in the task area, and the value is 0 if the reference track does not intersect with the obstacle in the task area, ωₐ, ωₛ and ωₗ are penalty weights corresponding to f_{α}, fₛ and fₗ, and when ω1 is 1 and ω2, ω3, ωa, ωs and ωl are 0, F indicates the length of a planned path; and
Step 2-3: after the robot moves according to the planned path to arrive at the target sampling point, performing sampling preparation work, including: turning on a sampling apparatus, checking whether a sensor state and a connection are normal and ensuring that the robot is at an appropriate posture and position to perform an accurate sampling action conveniently; and
Step 3: performing intelligent control sampling, including prediction analysis, optimization and control command output, including the following substeps:
Step 3-1: determining, by the robot, whether a soil sample to be extracted is required to be drilled and crushed according to a picture shot by vision sensing, skipping to Step 3-3 if the soil is soft, and skipping to Step 3-2 if a soil layer at the sampling point is hard;
Step 3-2: starting a drilling device to drill and crush the soil to be sampled;
Step 3-3: sending, by the control unit, a control signal to directly control a negative pressure suction mechanism to suck gas, liquid and solid multi-phase mixed components according to a maximum power;
Step 3-4: determining, by the robot, whether the extracted sample capacity meets the target, skipping to Step 3-6 if the target is met, and skipping to Step 3-5 if the target is not met;
Step 3-5: feeding, by the robot, a determination signal back to an intelligent control unit, restarting the drilling device and a negative pressure suction device, designing optimal drilling time, single-stage extraction time, an optimal extraction power and a drilling power, and performing Steps 3-1 to 3-4 again, where the optimization process includes the following substeps:
Step 3-5-1: achieving, by the robot, closed-loop dynamic control, and solving optimal control parameters before each stage of sample extraction, where the specific optimization process is as follows: the physical units of indexes are in brackets, a multi-target optimization equation is established with the lowest total cost sum of the power consumption cost of the drilling machine and the soil suction cost and with the shortest total time at the end of sampling of the sampling point:

$$\{ \begin{matrix}Min ES = S + E = {\sum }_{i = 1}^{{k}_{1}} {p}_{i} {t}_{i} + {\sum }_{j = 1}^{{k}_{2}} {p}_{j} {t}_{j} \\ Min t total = Max ( {\sum }_{i = 1}^{{k}_{1}} {t}_{i} , {\sum }_{j = 1}^{{k}_{2}} {t}_{j} )\end{matrix}$$

in the formula, S is a soil suction cost with the unit of watt, E is the power consumption cost of the drilling machine with the unit of watt, ES is the total cost sum with the unit of watt, pᵢ is the extraction power of a negative pressure extraction device within the i^{th} working time, with the unit of watt, pⱼ is the extraction power of a drilling system within the j^{th} working time, with the unit of watt, tᵢ is the time spent by the negative pressure extraction device in the i^{th} operation period, with the unit of second, tⱼ is the time spent by the drilling device in the j^{th} operation period, with the unit of second, t total is a total time at the end of sampling of the sampling point, with the unit of second, k₁ is the total number of starting times of the negative pressure extraction system, k₂ is the total number of starting times of the drilling system, with the constraint condition:

$${\sum }_{i = 1}^{{k}_{1}} {t}_{i} \leq T 1 m$$

T1m is the rated single extraction time of the negative pressure extraction system, with the unit of second,

$${\sum }_{j = 1}^{{k}_{2}} {t}_{j} \leq T 2 m$$

T2m is the rated single drilling time of the drilling system, with the unit of second,

$${\sum }_{i = 1}^{{k}_{1}} {m}_{i} \geq M$$

M is the target sampling quantity, and mi is the quantity of soil at a single negative pressure extraction, with the unit of gram;
Step 3-5-2: obtaining, by the controller, the optimal suction and drilling time, the extraction power and the drilling power in the next stage by solving the above equation, and transmitting a signal to an embedded control device, where a solving method is not limited to various algorithms including a genetic algorithm; and
Step 3-5-3: performing Steps 3-1 to 3-4 again; and
Step 3-6: ending sampling, and after completing the sampling task, transmitting, by the robot, the collected working condition data to a professional data storage system through a wireless network or a wired interface to perform recording.

Compared with the prior art, the present invention has the following beneficial effects:
1) according to the present invention, a vision sensing and radar sensing system is mounted in front of the robot body; when contaminated site sampling is required, the contaminated site can be positioned, information is transmitted to the PLC, and the servo motors controlled by the PLC to drive the track wheel to rotate, so that the robot can travel to the contaminated site autonomously; the drilling mechanism and the negative-pressure suction mechanism are controlled by the PLC to make the sample collecting pipe accurately arrive at the target position; and the drilled samples arrive at the sample collecting box through the pipeline under the drilling action of the drilling mechanism on the surface of the contaminated site, thereby implementing intelligent sampling and collection of the contaminated site.
2) One or more robots provided by the present invention can perform sampling cooperatively, a plurality of vision sensing and radar sensing systems may form a sensing network, a plurality of PLCs perform wireless communication, and sampling can be performed at different sampling points of the contaminated site at the same time during the sampling operation on the contaminated site, so that the sampling efficiency on the contaminated site can be improved. Furthermore, the robot can accurately identify and position the contaminated site, and also can plan the optimal path, effectively avoid obstacles and truly achieve the intelligent sampling on the contaminated site.
3) The robot provided by the present invention can supply power by a lithium battery or a direct-current generator. When the power supply adopts the direct-current generator, the power supply provided by the direct-current generator is stable, and the cruising ability is strong, thereby avoiding the problem of insufficient power of a mobile power supply. The lithium battery can be used as a backup power supply, so that the sampling robot can operate more stably and reliably.
4) When the intelligent sampling method for the contaminated site provided by the present invention is used for the intelligent sampling for the contaminated site, the looseness of the soil is determined through vision sensing, so that the device can be controlled to start and stop in time, and the sampling data is more accurate and efficient. Data is transmitted to the professional data storage system through the wireless network or the wired interface, which is convenient for workers to track and verify the data of the contaminated site.
5) According to the present invention, gas, liquid and solid components in the soil are sucked by a negative-pressure method at the same time, and multi-level information of the soil is captured, so that the loss of volatile substances can be effectively avoided, and the integrity of the sample components can be maintained. More comprehensive detection information can be provided for the subsequent soil sample analysis by obtaining the gas, liquid and solid components of the soil in different states.

### BRIEF DESCRIPTION OF DRAWINGS

FIG. 1 is a structural schematic diagram of a contaminated site sampling robot according to an embodiment of the present invention;

FIG. 2 is one of structural schematic diagrams of a contaminated site sampling robot according to an embodiment of the present invention;

FIG. 3 is a schematic diagram of a bottom structure of a contaminated site sampling robot according to an embodiment of the present invention;

FIG. 4 is a structural schematic diagram of a vision sensing system of a contaminated site sampling robot according to an embodiment of the present invention;

FIG. 5 is a structural schematic diagram of a drilling mechanism of a contaminated site sampling robot according to an embodiment of the present invention;

FIG. 6 is one of structural schematic diagrams of a drilling mechanism of a contaminated site sampling robot according to an embodiment of the present invention;

FIG. 7 is a structural schematic diagram of a negative-pressure suction mechanism of a contaminated site sampling robot according to an embodiment of the present invention; and

FIG. 8 is a schematic flowchart of an intelligent sampling method for a contaminated site sampling robot according to an embodiment of the present invention.

Reference numerals: 1. robot body; 11. walking mechanism; 111. mounting platform; 112. servo motor; 113. track wheel; 114. walking track; 12. vision sensing system; 121. supporting frame; 122. vision sensing camera; 123. radar sensor; 124. gear motor; 125. lead screw base; 126. double-thread lead screw; 127. lead screw nut; 128. guide rod; 13. drilling mechanism; 131. U-shaped base; 132. first supporting arm; 133. second supporting arm; 134. telescopic arm; 1341. rotating base; 135. drilling machine; 136. second electric telescopic rod; 137. third electric telescopic rod; 139. fourth electric telescopic rod; 14. negative-pressure suction mechanism; 141. U-shaped box; 142. vacuum cleaner; 143. sample collecting box; 144. pipeline; 145. sample collecting pipe; 146. box cover; 15. supporting mechanism; 151. first electric telescopic rod; 152. supporting pad; 2. battery box; 3. control box; 4. sample tank placing rack; 41. placing groove.

### DESCRIPTION OF THE EMBODIMENTS

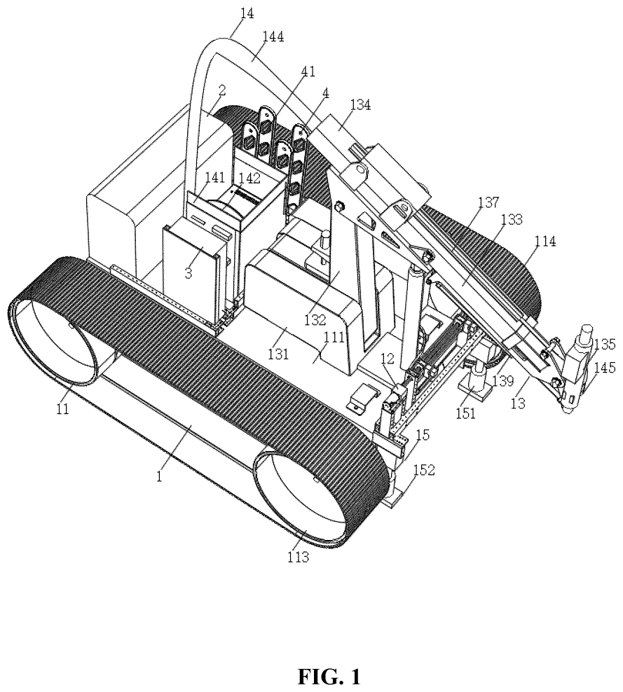

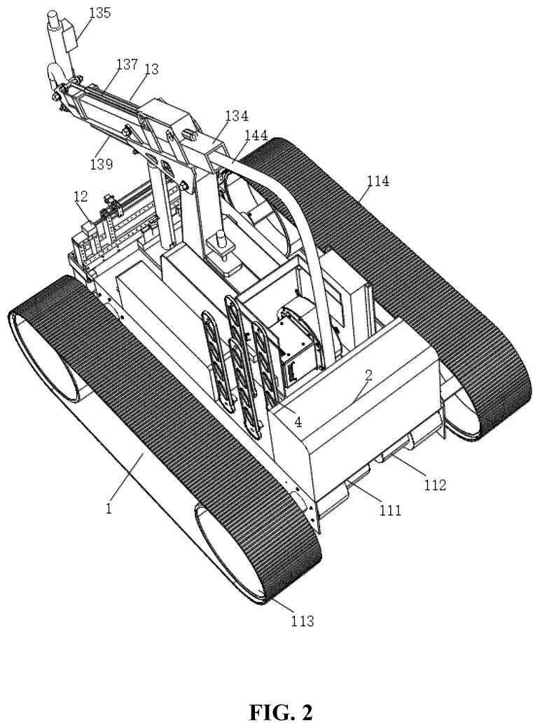

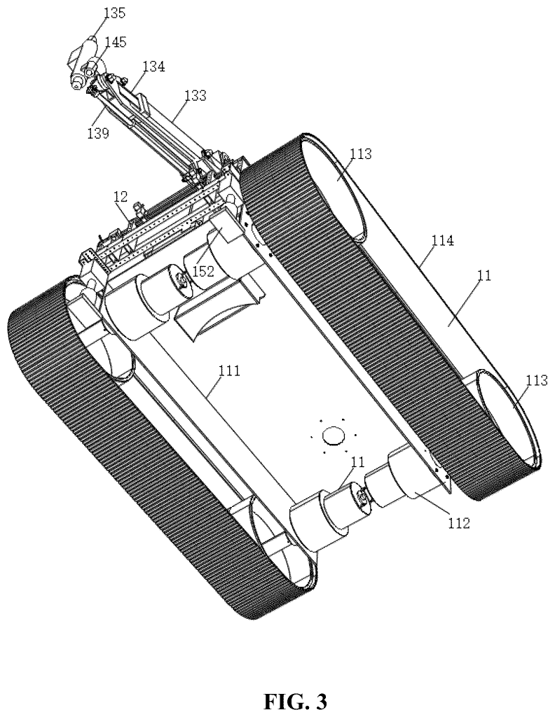

Referring to FIG. 1 to FIG. 3, a contaminated site sampling robot includes a robot body 1, where the robot body 1 includes a walking mechanism 11, a vision sensing system 12, a drilling mechanism 13 and a negative-pressure suction mechanism 14. The walking mechanism 11 includes a mounting platform 111, two servo motors 112 are fixedly mounted at two ends inside the mounting platform 111, a track wheel 113 is mounted at an output end of each of the servo motors 112, and a walking track 114 is sleeved on two track wheels 113 located on a same side; the vision sensing system 12 includes a supporting frame 121 mounted at the top of one end of the mounting platform 111, and a vision sensing camera 122 and a radar sensor 123 are arranged on one side of the top of the supporting frame 121; the drilling mechanism 13 includes a U-shaped base 131 mounted at the top of the mounting platform 111, an interior of one end of the U-shaped base 131 is hinged with a first supporting arm 132, the top of the first supporting arm 132 is hinged with a second supporting arm 133, a telescopic arm 134 is sleeved inside the second supporting arm 133, a rotating base 1341 is rotatably mounted at one end of the telescopic arm 134, and a drilling machine 135 is mounted on one side of the rotating base 1341; and the negative-pressure suction mechanism 14 includes a U-shaped box 141 mounted at the top end of a middle part of the mounting platform 111, a vacuum cleaner 142 is mounted inside the U-shaped box 141, a dust outlet of the vacuum cleaner 142 is connected to a sample collecting box 143, and a feeding port of the vacuum cleaner 142 is connected to a sample collecting pipe 145 through a pipeline 144.

The servo motors 112 may select the model number: Denmark JVL MAC400-D2-CAGM servo motor; the vision sensing camera 122 may select the model number: Germany SensoPart V20-OB-A1-W6 vision sensing camera; the radar sensor 123 may select the model number: Keli SDKELI LS2-1027BN/M05 radar sensor; the drilling machine 135 may select the model number: Germany IMT CLASSIC 33 A43 drilling machine; and the vacuum cleaner 142 may select the model number: Kardv DL-1032W vacuum cleaner.

The selection of the model number of each structural member in the present invention is not limited to the description of the specification of the present invention, and can be selected by those of ordinary skill in the art according to the actual situation and experience.

Specifically, the walking mechanism 11 can make the robot body 1 smoothly move to a contaminated site to be sampled; furthermore, each track wheel 113 is driven by a single servo motor 112, so that the robot body 1 can flexibly perform turning and differential control; the vision sensing system 12 can position the contaminated site and transmit information to the PLC; the servo motors 112 can be controlled by the PLC to drive the track wheels 113 to rotate, so that the robot can travel to the contaminated site autonomously; the walking track 114 can improve the off-road performance of the robot body 1; and when the robot body 1 moves to the contaminated site, the drilling mechanism 13 can drill the earth surface, and the negative-pressure suction mechanism 14 can suck the drilled sample into the sample collecting box 143 under negative pressure to collect samples.

The PLC may select the model number: Mitsubishi FX1NPLC.

In this embodiment, a supporting mechanism 15 is fixedly mounted at the bottom of the supporting frame 121, the supporting mechanism 15 includes two first electric telescopic rods 151 fixedly mounted at the bottom of the supporting frame 121, and supporting pads 152 are fixedly mounted at the bottoms of the first electric telescopic rods 151.

Specifically, when the robot body 1 samples the contaminated site, the first electric telescopic rods 151 of the supporting mechanism 15 extend out, and the supporting pads 152 are supported on the ground, thereby enhancing the stability of the robot body 1 placed on the ground and ensuring that the drilling mechanism 13 can drill the earth surface smoothly and stably.

In this embodiment, a battery box 2 is fixedly mounted at the top of the other end of the mounting platform 111, a control box 3 and a sample tank placing rack 4 which are respectively located on two sides of the U-shaped box 141 are further mounted at the top of the mounting platform 111, and a plurality of placing grooves 41 are formed in a surface of the sample tank placing rack 4.

A battery in the battery box 2 may select the model number: Germany RPOWER-BATTERY GIV1290H lead-acid storage battery.

Specifically, through the plurality of placing grooves 41 formed in the sample tank placing rack 4, the sample tank with samples collected can be taken out from the sample collecting box 143 and be placed and stored, so that the robot can perform diversified and flexible sampling on the contaminated site.

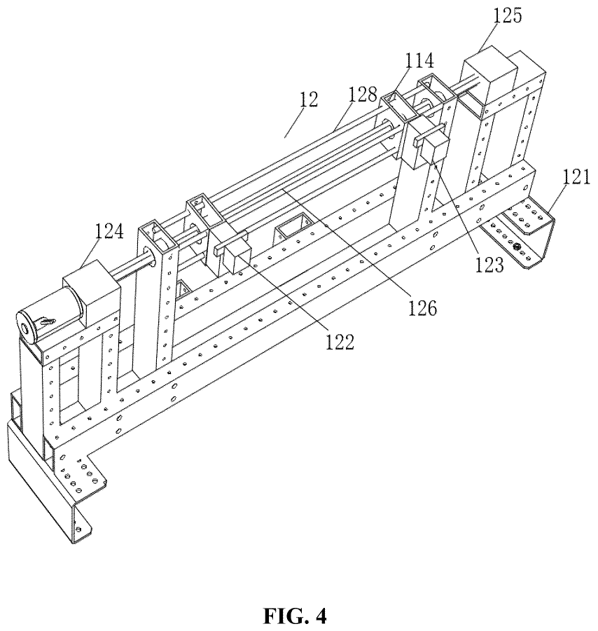

Referring to FIG. 4, the vision sensing system 12 further includes a gear motor 124 and a lead screw base 125 which are mounted at the tops of bosses at two ends of the supporting frame 121, a double-thread lead screw 126 is mounted at an output end of the gear motor 124, an exterior of the double-thread lead screw 126 is in threaded connection with two lead screw nuts 127, guide rods 128 are connected to middle parts of the two lead screw nuts 127 in an inserting manner, two ends of the guide rods 128 are fixedly connected to two bosses in a middle part of the supporting frame 121, and the vision sensing camera 122 and the radar sensor 123 are fixedly mounted on one side of each of the two lead screw nuts 127, respectively.

The gear motor 124 may select the model number: 5IK90RGU-CF/5GU3˜500K(YN90-90) gear motor.

Specifically, the gear motor 124 can drive the double-thread lead screw 126 to rotate, the rotating double-thread lead screw 126 can drive the two lead screw nuts 127 to perform reciprocating motion, the guide rods 128 can guide the motion process of the lead screw nuts 127, and the two lead rod nuts 127 that perform reciprocating motion can respectively drive the vision sensing camera 122 and the radar sensor 123 to perform reciprocating motion, so that the vision sensing system 12 can determine the terrain and position of the contaminated site and feed the terrain and position back to the PLC, and the PLC can control the walking mechanism 11 to move the robot body 1 to an accurate position.

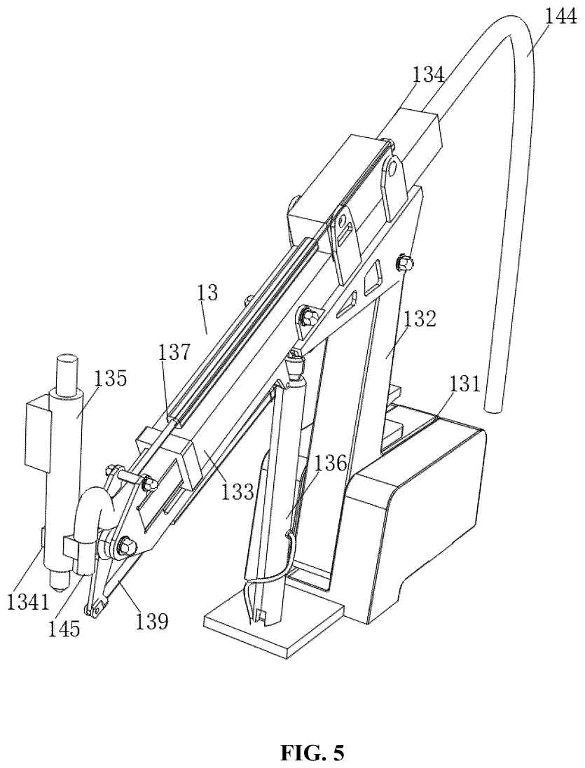

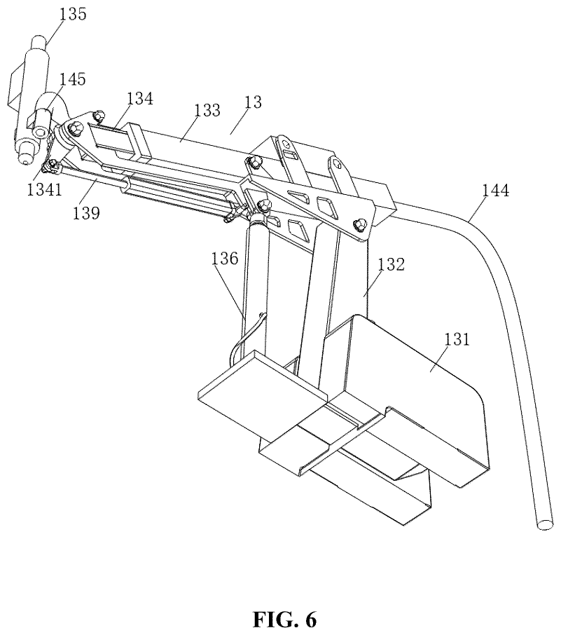

Referring to FIG. 5 to FIG. 6, the drilling mechanism 13 further includes a second electric telescopic rod 136 hinged at the bottom of the second supporting arm 133, the bottom of the second electric telescopic rod 136 is hinged with the top of a boss at one end of the U-shaped base 131, a third electric telescopic rod 137 is fixedly mounted at the top of the second supporting arm 133, one end of the third electric telescopic rod 137 is hinged with the top of the rotating base 1341, the bottom of one end of the rotating base 1341 is hinged with a fourth electric telescopic rod 139, the fourth electric telescopic rod 139 is mounted at the bottom of the second supporting arm 133 away from a telescopic end, the pipeline 144 is a corrugated pipe, and a middle part of the pipeline 144 is placed inside the hollow telescopic arm 134.

Specifically, the second electric telescopic rod 136 can drive the first supporting arm 132 to ascend and descend, the third electric telescopic rod 137 can drive the rotating base 1341 to move so as to make the telescopic arm 134 expand and contract, the expansion and contraction of the fourth electric telescopic rod 139 can drive the rotating base 1341 to rotate to change the angles of the drilling machine 135 and the sample collecting pipe 145, thereby ensuring the drilling machine 135 can drill an sample the earth surface; and the samples can be collected by the sample collecting pipe 145 under negative pressure, the pipeline 144 is placed inside the telescopic arm 134, and the pipeline 144 can be protected by the telescopic arm 134, so that the service life of the pipeline 144 can be prolonged.

The drilling machine 135 can select the model number: Germany IMT CLASSIC 33 A43 drilling machine.

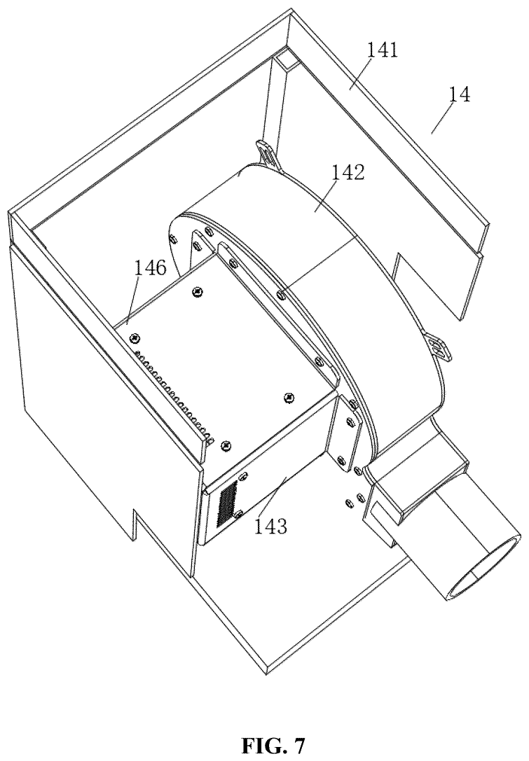

Referring to FIG. 7, the sample collecting box 143 is also mounted inside the U-shaped box 141, a sample tank communicating with the dust outlet of the vacuum cleaner 142 is arranged inside the sample connecting box 143, and the top of the sample collecting box 143 is hinged with a box cover 146.

Specifically, the sample tank can be taken and put conveniently by opening the box cover 146, and the sample tank can separately store the samples collected each time, so that the robot can sample the contaminated site for many times or respectively sample different areas; meanwhile, the samples are collected in a negative-pressure manner, so that the samples can be collected easily, dust can be effectively reduced, and the collection efficiency is more efficient than the sample collection efficiency in the traditional manner.

In this embodiment, the pipeline 144 is a corrugated pipe, and a middle part of the pipeline 144 is placed inside the hollow telescopic arm 134.

Specifically, the pipeline 144 is placed inside the telescopic arm 134, and the pipeline 144 can be protected by the telescopic arm 134, so that the service life of the pipeline 144 can be prolonged.

In this embodiment, the PLC is mounted inside the control box 3, the vision sensing camera 122 and the radar sensor 123 are electrically connected to the PLC, the PLC is electrically connected to the gear motor 124, the vacuum cleaner 142, the drilling machine 135, the first electric telescopic rods 151, the second electric telescopic rod 136, the third electric telescopic rod 137 and the fourth electric telescopic rod 139, and the PLC is electrically connected to a battery inside the battery box 2 and a direct-current generator.

Specifically, the PLC can respectively control the gear motor 124, the vacuum cleaner 142, the drilling machine 135, the first electric telescopic rods 151, the second electric telescopic rod 136, the third electric telescopic rod 137 and the fourth electric telescopic rod 139. One or more robots provided by the present invention can perform sampling cooperatively, a plurality of vision sensing and radar sensing systems may form a sensing network, a plurality of PLCs perform wireless communication, and sampling can be performed at different sampling points of the contaminated site at the same time during the sampling operation on the contaminated site, so that the sampling efficiency on the contaminated site can be improved. Furthermore, the robot can accurately identify and position the contaminated site, and also can plan the optimal path, effectively avoid obstacles and truly achieve the intelligent sampling on the contaminated site.

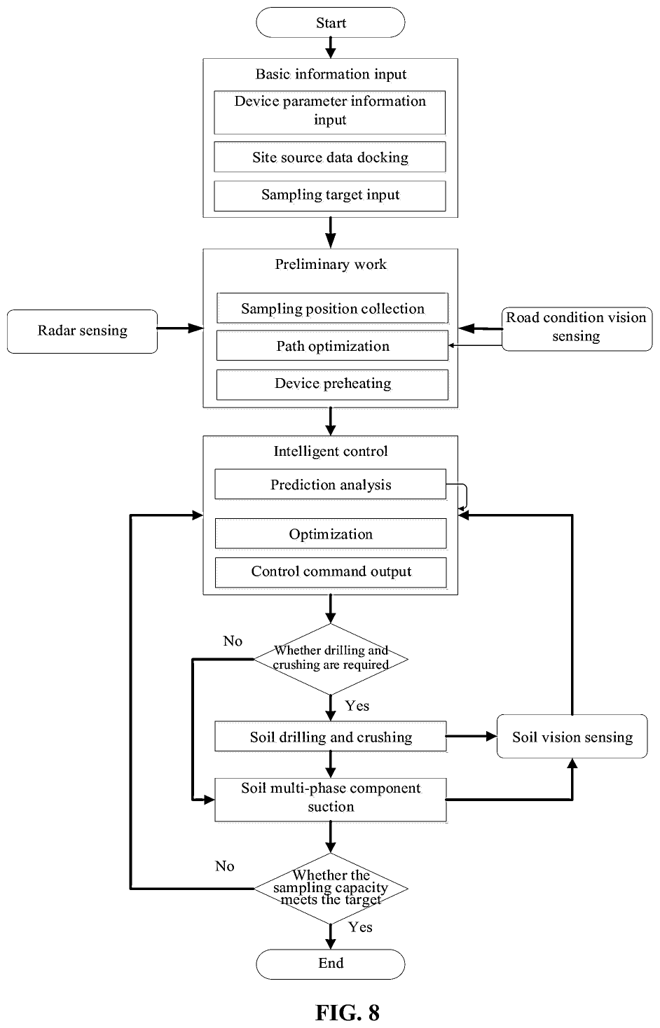

Referring to FIG. 8, an intelligent sampling method for a contaminated site uses the contaminated site sampling robot to collect soil, and specifically includes the following steps:
Step 1: basic information input is performed, including: device parameter information input, site resource data docking and sampling target input, including the following substeps:
Step 1-1: variables of the PLC, the vision sensing camera and the radar sensor are endowed as default values, and the gear motor, the vacuum cleaner, the drilling machine, the first electric telescopic rod, the second electric telescopic rod, the third electric telescopic rod and the fourth electric telescopic rod are set as default states;
Step 1-2: site environment information and space coordinate distribution information are input; and
Step 1-3: a distribution mode of sampling areas of the contaminated site and a sampling quantity requirement of each of the sampling areas are determined.
Step 2: preliminary work preparation is performed, including sampling position collection, path optimization and device preheating, including the following substeps:
Step 2-1: the robot surveys a site environment, and acquires a terrain, an obstacle position and air quality information of the site by a road condition vision sensor to provide reference for the subsequent navigation and sampling;
Step 2-2: the robot performs self-positioning and surrounding environment perception by a radar sensor, constructs a map by an SLAM technology and updates the own position; and a control unit considers whether factors such as a maximum corner and a maximum power stroke of the robot are met according to a preset robot starting point and a target sampling point and in combination with map data and obstacle information, and determines a best path to a sampling point by an intelligent sampling algorithm, where a robot path planning fitness function is:

$$F = {\sum }_{i = 1}^{n} ( {ω}_{1} \cdot {l}_{i} + {ω}_{2} \cdot {h}_{i} + {ω}_{3} \cdot {α}_{i} ) + ( {ω}_{a} \cdot {f}_{α} + {ω}_{s} \cdot {f}_{s} + {ω}_{l} \cdot {f}_{l} )$$

in the formula, lᵢ is the length of a i^{th} reference path, hᵢ is the height difference of the i^{th} reference path, αᵢ is the smoothness of the i^{th} reference path, ω₁, ω₂ and ω₃ are respectively weights of the length, the height difference and the smoothness, f_{α} indicates whether the steering angle of the reference path exceeds a maximum steering angle, the value is 1 if the steering angle of the reference path exceeds the maximum steering angle, and the value is 0 if the steering angle of the reference path does not exceed the maximum steering angle, fₛ indicates whether the reference path exceeds a power range, the value is 1 if the reference path exceeds the power range, and the value is 0 if the reference path does not exceed the power range, fₗ indicates whether a reference track intersects with an obstacle in a task area, the value is 1 if the reference track intersects with the obstacle in the task area, and the value is 0 if the reference track does not intersect with the obstacle in the task area, ωₐ, ωₛ and ωₗ are penalty weights corresponding to f_{α}, fₛ and fₗ, and when ω1 is 1 and ω2, ω3, ωa, ωs and ωl are 0, F indicates the length of a planned path; and
Step 2-3: after moving according to the planned path to arrive at the target sampling point, the robot performs sampling preparation work, including: turning on a sampling apparatus, checking whether a sensor state and a connection are normal and ensuring that the robot is at an appropriate posture and position to perform an accurate sampling action.
Step 3: intelligent control sampling is performed, including prediction analysis, optimization and control command output, including the following substeps:
Step 3-1: the robot determines whether a soil sample to be extracted is required to be drilled and crushed according to a picture shot by vision sensing, the step is skipped to Step 3-3 if the soil is soft, and the step is skipped to Step 3-2 if a soil layer at the sampling point is hard;
Step 3-2: a drilling device is started to drill and crush the soil to be sampled;
Step 3-3: the control unit sends a control signal to directly control a negative pressure suction mechanism to suck gas, liquid and solid multi-phase mixed components according to a maximum power;
Step 3-4: the robot determines whether the extracted sample capacity meets the target, skipping to Step 3-6 if the target is met, and skipping to Step 3-5 if the target is not met;
Step 3-5: the robot feeds a determination signal back to an intelligent control unit, the drilling device and a negative pressure suction device are restarted, optimal drilling time, single-stage extraction time, an optimal extraction power and a drilling power are designed, and Steps 3-1 to 3-4 are performed again, where the optimization process includes the following substeps:
Step 3-5-1: the robot can achieve closed-loop dynamic control, and optimal control parameters are solved before each stage of sample extraction, where the specific optimization process is as follows: the physical units of indexes are in brackets, a multi-target optimization equation is established with the lowest total cost sum of the power consumption cost of the drilling machine and the soil suction cost and with the shortest total time at the end of sampling of the sampling point:

$$\{ \begin{matrix}Min ES = S + E = {\sum }_{i = 1}^{{k}_{1}} {p}_{i} {t}_{i} + {\sum }_{j = 1}^{{k}_{2}} {p}_{j} {t}_{j} \\ Min t total = Max ( {\sum }_{i = 1}^{{k}_{1}} {t}_{i} , {\sum }_{j = 1}^{{k}_{2}} {t}_{j} )\end{matrix}$$

in the formula, S is a soil suction cost with the unit of watt, E is the power consumption cost of the drilling machine with the unit of watt, ES is the total cost sum with the unit of watt, pᵢ is the extraction power of a negative pressure extraction device within the i^{th} working time, with the unit of watt, pⱼ is the extraction power of a drilling system within the j^{th} working time, with the unit of watt, tᵢ is the time spent by the negative pressure extraction device in the i^{th} operation period, with the unit of second, tⱼ is the time spent by the drilling device in the j^{th} operation period, with the unit of second, t total is a total time at the end of sampling of the sampling point, with the unit of second, k₁ is the total number of starting times of the negative pressure extraction system, k₂ is the total number of starting times of the drilling system, with the constraint condition:

$${\sum }_{i = 1}^{{k}_{1}} {t}_{i} \leq T 1 m$$

T1m is the rated single extraction time of the negative pressure extraction system, with the unit of second,

$${\sum }_{i = 1}^{{k}_{2}} {t}_{j} \leq T 2 m$$

T2m is the rated single drilling time of the drilling system, with the unit of second,

$${\sum }_{i = 1}^{{k}_{1}} {m}_{i} \geq M$$

M is the target sampling quantity, and mi is the quantity of soil at a single negative pressure extraction, with the unit of gram;
Step 3-5-2: the controller obtains the optimal suction and drilling time, the extraction power and the drilling power in the next stage by solving the above equation, and transmits a signal to an embedded control device, where aa solving method is not limited to various algorithms including a genetic algorithm; and
Step 3-5-3: Steps 3-1 to 3-4 again are performed; and
Step 3-6: sampling is ended, and after the sampling task is completed, the robot transmits the collected working condition data to a professional data storage system through a wireless network or a wired interface to perform recording.

What is claimed is:

1. A contaminated site sampling robot, comprising a robot body (1), wherein the robot body (1) comprises a walking mechanism (11), a vision sensing system (12), a drilling mechanism (13), and a negative-pressure suction mechanism (14); the walking mechanism (11) comprises a mounting platform (111), two servo motors (112) are fixedly mounted at two ends inside the mounting platform (111), a track wheel (113) is mounted at an output end of each of the servo motors (112), and a walking track (114) is sleeved on two track wheels (113) located on a same side; the vision sensing system (12) comprises a supporting frame (121) mounted at the top of one end of the mounting platform (111), and a vision sensing camera (122) and a radar sensor (123) are arranged on one side of the top of the supporting frame (121); the drilling mechanism (13) comprises a U-shaped base (131) mounted at the top of the mounting platform (111), an interior of one end of the U-shaped base (131) is hinged with a first supporting arm (132), the top of the first supporting arm (132) is hinged with a second supporting arm (133), a telescopic arm (134) is sleeved inside the second supporting arm (133), a rotating base (1341) is rotatably mounted at one end of the telescopic arm (134), and a drilling machine (135) is mounted on one side of the rotating base (1341); and the negative-pressure suction mechanism (14) comprises a U-shaped box (141) mounted at a top end of a middle part of the mounting platform (111), a vacuum cleaner (142) is mounted inside the U-shaped box (141), a dust outlet of the vacuum cleaner (142) is connected to a sample collecting box (143), and a feeding port of the vacuum cleaner (142) is connected to a sample collecting pipe (145) through a pipeline (144).

2. The contaminated site sampling robot according to claim 1, wherein a supporting mechanism (15) is fixedly mounted at the bottom of the supporting frame (121), the supporting mechanism (15) comprises two first electric telescopic rods (151) fixedly mounted at the bottom of the supporting frame (121), and supporting pads (152) are fixedly mounted at the bottoms of the first electric telescopic rods (151).

3. The contaminated site sampling robot according to claim 2, wherein a battery box (2) is fixedly mounted at the top of the other end of the mounting platform (111), a control box (3) and a sample tank placing rack (4) which are respectively located on two sides of the U-shaped box (141) are further mounted at the top of the mounting platform (111), and a plurality of placing grooves (41) are formed in a surface of the sample tank placing rack (4).

4. The contaminated site sampling robot according to claim 3, wherein the vision sensing system (12) further comprises a gear motor (124) and a lead screw base (125) which are mounted at the tops of bosses at two ends of the supporting frame (121), a double-thread lead screw (126) is mounted at an output end of the gear motor (124), an exterior of the double-thread lead screw (126) is in threaded connection with two lead screw nuts (127), guide rods (128) are connected to middle parts of the two lead screw nuts (127) in an inserting manner, two ends of the guide rods (128) are fixedly connected to two bosses in a middle part of the supporting frame (121), and the vision sensing camera (122) and the radar sensor (123) are fixedly mounted on one side of each of the two lead screw nuts (127), respectively.

5. The contaminated site sampling robot according to claim 4, wherein the drilling mechanism (13) further comprises a second electric telescopic rod (136) hinged at the bottom of the second supporting arm (133), the bottom of the second electric telescopic rod (136) is hinged with the top of a boss at one end of the U-shaped base (131), a third electric telescopic rod (137) is fixedly mounted at the top of the second supporting arm (133), one end of the third electric telescopic rod (137) is hinged with the top of the rotating base (1341), the bottom of one end of the rotating base (1341) is hinged with a fourth electric telescopic rod (139), and the fourth electric telescopic rod (139) is mounted at the bottom of the second supporting arm (133) away from a telescopic end.

6. The contaminated site sampling robot according to claim 1, wherein the sample collecting box (143) is also mounted inside the U-shaped box (141), a sample tank communicating with the dust outlet of the vacuum cleaner (142) is arranged inside the sample collecting box (143), and the top of the sample collecting box (143) is hinged with a box cover (146).

7. The contaminated site sampling robot according to claim 3, wherein the pipeline (144) is a corrugated pipe, and a middle part of the pipeline (144) is placed inside the hollow telescopic arm (134).

8. The contaminated site sampling robot according to claim 5, wherein a PLC is mounted inside the control box (3), the vision sensing camera (122) and the radar sensor (123) are electrically connected to the PLC, the PLC is electrically connected to the gear motor (124), the vacuum cleaner (142), the drilling machine (135), the first electric telescopic rod (151), the second electric telescopic rod (136), the third electric telescopic rod (137) and the fourth electric telescopic rod (139), and the PLC is electrically connected to a battery inside the battery box (2) and a direct-current generator.

9. An intelligent sampling method for a contaminated site, using the contaminated site sampling robot according to claim 5 to collect soil, and specifically comprising the following steps:
Step 1: performing basic information input, comprising: device parameter information input, site resource data docking, and sampling target input, comprising the following substeps:
Step 1-1: endowing variables of the PLC, the vision sensing camera and the radar sensor as default values, and setting the gear motor, the vacuum cleaner, the drilling machine, the first electric telescopic rod, the second electric telescopic rod, the third electric telescopic rod and the fourth electric telescopic rod as default states;
Step 1-2: inputting site environment information and space coordinate distribution information; and
Step 1-3: determining a distribution mode of sampling areas of the contaminated site and a sampling quantity requirement of each of the sampling areas;
Step 2: performing preliminary work preparation, comprising sampling position collection, path optimization, and device preheating, comprising the following substeps:
Step 2-1: surveying, by the robot, a site environment, and acquiring a terrain, an obstacle position and air quality information of the site by a road condition vision sensor to provide reference for the subsequent navigation and sampling;
Step 2-2: performing, by the robot, self-positioning and surrounding environment perception by the radar sensor, constructing a map by an SLAM technology and updating the own position, considering, by a control unit, whether factors such as a maximum corner and a maximum power stroke of the robot are met according to a preset robot starting point and a target sampling point and in combination with map data and obstacle information, and determining a best path to a sampling point by an intelligent sampling algorithm, wherein a robot path planning fitness function is:
$$F = {\sum }_{i = 1}^{n} ( {ω}_{1} \cdot {l}_{i} + {ω}_{2} \cdot {h}_{i} + {ω}_{3} \cdot {α}_{i} ) + ( {ω}_{a} \cdot {f}_{α} + {ω}_{s} \cdot {f}_{s} + {ω}_{l} \cdot {f}_{l} )$$
in the formula, lᵢ is the length of a i^{th} reference path, hᵢ is the height difference of the i^{th} reference path, α₁ is the smoothness of the i^{th} reference path, ω₁, ω₂ and ω₃ are respectively weights of the length, the height difference and the smoothness, f_{α} indicates whether the steering angle of the reference path exceeds a maximum steering angle, the value is 1 if the steering angle of the reference path exceeds the maximum steering angle, and the value is 0 if the steering angle of the reference path does not exceed the maximum steering angle, fₛ indicates whether the reference path exceeds a power range, the value is 1 if the reference path exceeds the power range, and the value is 0 if the reference path does not exceed the power range, fₗ indicates whether a reference track intersects with an obstacle in a task area, the value is 1 if the reference track intersects with the obstacle in the task area, and the value is 0 if the reference track does not intersect with the obstacle in the task area, ωₐ, ωₛ and ωₗ are penalty weights corresponding to f_{α}, fₛ and fₗ, and when ω1 is 1 and ω2, ω3, ωa, ωs and ωl are 0, F indicates the length of a planned path; and
Step 2-3: after the robot moves according to the planned path to arrive at the target sampling point, performing sampling preparation work, comprising: turning on a sampling apparatus, checking whether a sensor state and a connection are normal and ensuring that the robot is at an appropriate posture and position to perform an accurate sampling action; and
Step 3: performing intelligent control sampling, comprising prediction analysis, optimization, and control command output, comprising the following substeps:
Step 3-1: determining, by the robot, whether a soil sample to be extracted is required to be drilled and crushed according to a picture shot by vision sensing, skipping to Step 3-3 if the soil is soft, and skipping to Step 3-2 if a soil layer at the sampling point is hard;
Step 3-2: starting a drilling device to drill and crush soil to be sampled;
Step 3-3: sending, by the control unit, a control signal to directly control the negative-pressure suction mechanism to suck gas, liquid and solid multi-phase mixed components according to a maximum power;
Step 3-4: determining, by the robot, whether the extracted sample capacity meets the target, skipping to Step 3-6 if the target is met, and skipping to Step 3-5 if the target is not met;
Step 3-5: feeding, by the robot, a determination signal back to an intelligent control unit, restarting the drilling device and a negative-pressure suction device, designing optimal drilling time, single-stage extraction time, an optimal extraction power and a drilling power, and performing Steps 3-1 to 3-4 again, wherein the optimization process comprises the following substeps:
Step 3-5-1: achieving, by the robot, closed-loop dynamic control, and solving optimal control parameters before each stage of sample extraction, wherein the specific optimization process is as follows: the physical units of indexes are in brackets, a multi-target optimization equation is established with the lowest total cost sum of the power consumption cost of the drilling machine and the soil suction cost and with the shortest total time at the end of sampling of the sampling point:
$$\{ \begin{matrix}Min ES = S + E = {\sum }_{i = 1}^{{k}_{1}} {p}_{i} {t}_{i} + {\sum }_{j = 1}^{{k}_{2}} {p}_{j} {t}_{j} \\ Min t total = Max ( {\sum }_{i = 1}^{{k}_{1}} {t}_{i} , {\sum }_{j = 1}^{{k}_{2}} {t}_{j} )\end{matrix}$$
in the formula, S is the soil suction cost, with the unit of watt, E is the power consumption cost of the drilling machine, with the unit of watt, ES is the total cost sum, with the unit of watt, pᵢ is the extraction power of a negative-pressure extraction device within the i^{th} working time, with the unit of watt, pⱼ is the extraction power of a drilling system within the j^{th} working time, with the unit of watt, tᵢ is the time spent by the negative-pressure extraction device in the i^{th} operation period, with the unit of second, tⱼ is the time spent by the drilling device in the j^{th} operation period, with the unit of second, t total is a total time at the end of sampling of the sampling point, with the unit of second, k₁ is the total number of starting times of the negative-pressure extraction system, k₂ is the total number of starting times of the drilling system, with the constraint condition:
$${\sum }_{i = 1}^{{k}_{1}} {t}_{i} \leq T 1 m$$
T1m is the rated single extraction time of the negative-pressure extraction system, with the unit of second,
$${\sum }_{j = 1}^{{k}_{2}} {t}_{j} \leq T 2 m$$
T2m is the rated single drilling time of the drilling system, with the unit of second,
$${\sum }_{i = 1}^{{k}_{1}} {m}_{i} \geq M$$
M is the target sampling quantity, and mi is the quantity of soil at a single negative-pressure extraction, with the unit of gram;
Step 3-5-2: obtaining, by the controller, the optimal suction and drilling time, the extraction power and the drilling power in the next stage by solving the above equation, and transmitting a signal to an embedded control device, wherein a solving method is not limited to various algorithms comprising a genetic algorithm; and
Step 3-5-3: performing Steps 3-1 to 3-4 again; and
Step 3-6: ending sampling, and after completing the sampling task, transmitting, by the robot, the collected working condition data to a professional data storage system through a wireless network or a wired interface to perform recording.

10. An intelligent sampling method for a contaminated site, using the contaminated site sampling robot according to claim 8 to collect soil, and specifically comprising the following steps:
Step 1: performing basic information input, comprising: device parameter information input, site resource data docking, and sampling target input, comprising the following substeps:
Step 1-1: endowing variables of the PLC, the vision sensing camera and the radar sensor as default values, and setting the gear motor, the vacuum cleaner, the drilling machine, the first electric telescopic rod, the second electric telescopic rod, the third electric telescopic rod and the fourth electric telescopic rod as default states;
Step 1-2: inputting site environment information and space coordinate distribution information; and
Step 1-3: determining a distribution mode of sampling areas of the contaminated site and a sampling quantity requirement of each of the sampling areas;
Step 2: performing preliminary work preparation, comprising sampling position collection, path optimization, and device preheating, comprising the following substeps:
Step 2-1: surveying, by the robot, a site environment, and acquiring a terrain, an obstacle position and air quality information of the site by a road condition vision sensor to provide reference for the subsequent navigation and sampling;
Step 2-2: performing, by the robot, self-positioning and surrounding environment perception by the radar sensor, constructing a map by an SLAM technology and updating the own position, considering, by a control unit, whether factors such as a maximum corner and a maximum power stroke of the robot are met according to a preset robot starting point and a target sampling point and in combination with map data and obstacle information, and determining a best path to a sampling point by an intelligent sampling algorithm, wherein a robot path planning fitness function is:
$$F = {\sum }_{i = 1}^{n} ( {ω}_{1} \cdot {l}_{i} + {ω}_{2} \cdot {h}_{i} + {ω}_{3} \cdot {α}_{i} ) + ( {ω}_{a} \cdot {f}_{α} + {ω}_{s} \cdot {f}_{s} + {ω}_{l} \cdot {f}_{l} )$$
in the formula, lᵢ is the length of a i^{th} reference path, hᵢ is the height difference of the i^{th} reference path, α₁ is the smoothness of the i^{th} reference path, ω₁, ω₂ and ω₃ are respectively weights of the length, the height difference and the smoothness, f_{α} indicates whether the steering angle of the reference path exceeds a maximum steering angle, the value is 1 if the steering angle of the reference path exceeds the maximum steering angle, and the value is 0 if the steering angle of the reference path does not exceed the maximum steering angle, fₛ indicates whether the reference path exceeds a power range, the value is 1 if the reference path exceeds the power range, and the value is 0 if the reference path does not exceed the power range, fi indicates whether a reference track intersects with an obstacle in a task area, the value is 1 if the reference track intersects with the obstacle in the task area, and the value is 0 if the reference track does not intersect with the obstacle in the task area, ωₐ, ωₛ and ωₗ are penalty weights corresponding to f_{α}, fₛ and fₗ, and when ω1 is 1 and ω2, ω3, ωa, ωs and ωl are 0, F indicates the length of a planned path; and
Step 2-3: after the robot moves according to the planned path to arrive at the target sampling point, performing sampling preparation work, comprising: turning on a sampling apparatus, checking whether a sensor state and a connection are normal and ensuring that the robot is at an appropriate posture and position to perform an accurate sampling action; and
Step 3: performing intelligent control sampling, comprising prediction analysis, optimization, and control command output, comprising the following substeps:
Step 3-1: determining, by the robot, whether a soil sample to be extracted is required to be drilled and crushed according to a picture shot by vision sensing, skipping to Step 3-3 if the soil is soft, and skipping to Step 3-2 if a soil layer at the sampling point is hard;
Step 3-2: starting a drilling device to drill and crush soil to be sampled;
Step 3-3: sending, by the control unit, a control signal to directly control the negative-pressure suction mechanism to suck gas, liquid and solid multi-phase mixed components according to a maximum power;
Step 3-4: determining, by the robot, whether the extracted sample capacity meets the target, skipping to Step 3-6 if the target is met, and skipping to Step 3-5 if the target is not met;
Step 3-5: feeding, by the robot, a determination signal back to an intelligent control unit, restarting the drilling device and a negative-pressure suction device, designing optimal drilling time, single-stage extraction time, an optimal extraction power and a drilling power, and performing Steps 3-1 to 3-4 again, wherein the optimization process comprises the following substeps:
Step 3-5-1: achieving, by the robot, closed-loop dynamic control, and solving optimal control parameters before each stage of sample extraction, wherein the specific optimization process is as follows: the physical units of indexes are in brackets, a multi-target optimization equation is established with the lowest total cost sum of the power consumption cost of the drilling machine and the soil suction cost and with the shortest total time at the end of sampling of the sampling point:
$$\{ \begin{matrix}Min ES = S + E = {\sum }_{i = 1}^{{k}_{1}} {p}_{i} {t}_{i} + {\sum }_{j = 1}^{{k}_{2}} {p}_{j} {t}_{j} \\ Min t total = Max ( {\sum }_{i = 1}^{{k}_{1}} {t}_{i} , {\sum }_{j = 1}^{{k}_{2}} {t}_{j} )\end{matrix}$$
in the formula, S is the soil suction cost, with the unit of watt, E is the power consumption cost of the drilling machine, with the unit of watt, ES is the total cost sum, with the unit of watt, pᵢ is the extraction power of a negative-pressure extraction device within the i^{th} working time, with the unit of watt, pⱼ is the extraction power of a drilling system within the j^{th} working time, with the unit of watt, tᵢ is the time spent by the negative-pressure extraction device in the i^{th} operation period, with the unit of second, tⱼ is the time spent by the drilling device in the j^{th} operation period, with the unit of second, t total is a total time at the end of sampling of the sampling point, with the unit of second, k₁ is the total number of starting times of the negative-pressure extraction system, k₂ is the total number of starting times of the drilling system, with the constraint condition:
$${\sum }_{i = 1}^{{k}_{1}} {t}_{i} \leq T 1 m$$
T1m is the rated single extraction time of the negative-pressure extraction system, with the unit of second,
$${\sum }_{j = 1}^{{k}_{2}} {t}_{j} \leq T 2 m$$
T2m is the rated single drilling time of the drilling system, with the unit of second,
$${\sum }_{i = 1}^{{k}_{1}} {m}_{i} \geq M$$
M is the target sampling quantity, and mi is the quantity of soil at a single negative-pressure extraction, with the unit of gram;
Step 3-5-2: obtaining, by the controller, the optimal suction and drilling time, the extraction power and the drilling power in the next stage by solving the above equation, and transmitting a signal to an embedded control device, wherein a solving method is not limited to various algorithms comprising a genetic algorithm; and
Step 3-5-3: performing Steps 3-1 to 3-4 again; and
Step 3-6: ending sampling, and after completing the sampling task, transmitting, by the robot, the collected working condition data to a professional data storage system through a wireless network or a wired interface to perform recording.
